(19) United States
(12) Patent Application Publication
(10) Pub. No.: US 2007/0135749 A1
(43) Pub. Date: Jun. 14, 2007

(54) Response of lower-leg extremities

(76) Inventors: Emmanuel R. Williams, Park Forest, IL (US); Raymond N. Baker, Washington, DC (US)

Correspondence Address: Shanley and Baker, P.L.L.C..;Raymond N. Baker, P.O. Box 9995, Washington, DC, 20016-8995, US

(21) Appl. No.: 11/634,679

(22) Filed: Dec. 6, 2006

Related U.S. Application Data
(60) Provisional application No. 60/748,424, filed on Dec. 8, 2005.

(51) Int. Cl. A61F 13/06; A61F 5/37; A61F 13/00

(52) U.S. Cl. 602/62; 128/882

(57) ABSTRACT

Therapeutic-sock, method of fabricating, and method of use to relieve difficulties in walking about and discomfort in the lower leg extremities, following time-periods of immobility. Such therapeutic-sock is fabricated from a combination of thick and thin elasticized fabrics. Circumscribing compressed-forces are exerted on the lower-leg extremities so as it initiate and directionally continue return of depleted-fluids from the lower-leg extremities toward the heart; while facilitating arterial supply of oxygen and nutrients to such lower-leg extremities.

### RELATED APPLICATION

This application claims the benefit of U.S. Provisional Patent Application Ser. No. 60/748,424 filed Dec. 8, 2005.

### INTRODUCTION

This invention relates to improving response in the lower-leg extremities for an individual during and after a period of immobility. More particularly, this invention is concerned with eliminating discomfort associated with lower-leg extremities for individuals who remain substantially immobile when it is not permissible or appropriate to walk-freely about.

### OBJECTS OF THE INVENTION

A primary object is to help prevent pain, discomfort and possible swelling associated with the lower-leg extremities of certain individuals, for example: during an extended air flight when such a passenger may be required to remain seated.

An important object is concerned with properly analyzing causation in order to facilitate properly avoiding substantial loss of agility in the leg muscles of such an individual.

A related object is to help prevent such difficulties when, whether voluntary or involuntary, a person's situation provides little or no opportunity to walk-freely about, for a time-interval which is detrimental to normal functioning of return-capillaries in body-tissues of such a person's lower-leg extremities.

A further related object is to help achieve and maintain proper replenishment of oxygen and nutrients in the lower-leg extremities so as to facilitate normal functioning.

A specific object is to achieve a balanced-relationship in supply and return types of capillaries, associated with interstitial fluids of body-tissues of the lower-leg extremities.

Other objects and a fuller understanding, of the invention are made available by referring to the following description; and to the claims, which are to be interpreted based on the present disclosure and description in conjunction with the accompanying drawings.

### BRIEF DESCRIPTION OF FIGURES

FIG. 1 is a schematic perspective view of an individual's foot and lower leg for identifying placement-areas located for proper usage of circumscribing compressive-forces generated by a therapeutic sock of the invention.

FIG. 2 is a view in elevation of an sock-embodiment of the invention showing areas for two functional types of fabric, when such sock is not in use, for describing (i) initiating and (ii) continuing sequentially-directed circumscribing compressive-forces onto specifically lower-leg extremity placement-areas; while (iii) specifically limiting leg-height compressive-force usage in accordance with the invention;

FIG. 3(a) is an enlarged detailed external-surface elevational view of a minor portion, at a selected placement area, of thick elasticized-fabric of the invention, when the sock-embodiment of the invention is not in use; while

FIG. 3(b) is an enlarged detailed external-surface elevational view for describing expansion, in accordance with the invention, of such minor portion of the fabric shown in FIG. 3(a), during usage on such selected placement-area of a lower-leg extremity of an individual user;

FIG. 3(c) is an interior surface view of the FIG. 3(b) expanded thick-fabric portion of an elasticized sock of the invention for therapeutically-augmenting return-circulation capabilities, in particular by means of expanded, open-faced, widened-channels which confront selected lower-leg extremity placement-areas of an individual user, and

FIG. 3(d) is a cross-sectional view of a portion of FIG. 3(c), taken in a plane, which is perpendicular to the interior centerline for circumscribing conference-forces, for describing selectively-minimized leg-surface contact, in accordance with the invention, for supporting such open-faced widened-channels of FIG. 3(c), for augmenting return of depleted fluids for re-vitalization.

### DETAILED DESCRIPTION OF THE INVENTION

At varying ages, more generally in approaching or during seniority, individuals can experience difficulties associated with the lower-leg extremities. Such difficulties can be pronounced when such an individual attempts to walk-about following a relevant period of immobility whether voluntary or involuntary. A relevant time-period can be about twenty-five to forty-five minutes of seated immobility.

Such difficulties are manifested when such an individual first attempts to walk about following such a period of immobility. For example, the first of several twenty to thirty feet lengths of unaided walking, following immobility, are unsteady; and, more appropriately, could be described as “hobbling” about, while such an individual attempts to restore more-normal agility to his or her leg muscles.

The objectives and therapeutic functional purposes of the invention are to: (I) initiate help to prevent swelling in the lower-leg extremities, (ii) help to maintain normal tone in leg muscles of a user, by (iii) properly directing circumscribing compressive forces of the invention, to (iv) prevent such occurrences from occurring following such periods of immobility.

Capillaries carryout functional purposes in body-tissues resulting from the pumping action of the heart. Arterial-supplied blood is substantially reduced in pressure as such blood enters body-tissue capillaries of the lower-leg extremities. However, arterial-supported blood pressure enables filtering through thin walled entry capillaries of the lower-leg extremities; into interstitial fluids of body-tissues of such extremities. Carbon-dioxide, and other wastes, move from cells of those body-tissues into such interstitial-fluids.

Carbon-dioxide and other wastes move into interstitial-fluids of body-tissues of individuals referred to herein. That take-up of wastes can diminish capabilities, of such individuals to return depleted fluids from lower-leg extremities.

Lower-leg extremity difficulties, for such individuals, which result largely from immobility, can be compounded by swelling surrounding body-tissues of the lower-leg extremities. Long air flights accompanied by an absence of the opportunity to walk-freely about, can cause such swelling, which inhibits intended return-circulation of depleted body-fluids from the lower-leg extremities toward the heart.

Capillaries and associated interstitial fluids are the body's main system for distributing oxygen and nutrients, where needed, throughout the body. Thick-walled arteries carry oxygen and nutrient-rich blood, pumped by the heart, to all areas of the human body. Those thick-walled arteries branch repeatedly in the long travel toward the lower-leg extremities; those arteries becoming smaller and smaller, forming arterioles, in approaching areas for utilization of such oxygen and nutrients. At those areas, of tiny capillaries, which are only about a single cell thick, carry oxygen and nutrients to cells of the body tissues. The lower-leg extremities use such oxygen and nutrients metabolically, largely in a chemical metamorphosis which should proceed substantially continuously with the rate of such metamorphosis changing as needed by an individual.

The present invention is concerned with overcoming difficulties in returning such depleted interstitial-fluids, plus wastes, so as to enable and augment proper metabolic action in body-tissues of the lower-leg extremities. The intended function of return capillaries of the lower-leg extremities is to return depleted-fluids, containing resulting wastes, from the lower-leg extremities. That desirable function can be impeded in the lower-leg extremities, for the above-described individuals; swelling in such extremities due to immobility.

The functions of the therapeutic sock of the invention are to initiate and to facilitate return of such depleted body-fluids, and wastes, toward locations providing for return toward the heart. Accomplishing such return action, in itself, helps to prevent swelling in the lower-leg extremities. The elasticized fabrics of the invention apply circumscribing compressive-forces (i) starting at the forward leading-edge of the foot-arch; and, as importantly, (ii) directionally-continuing such circumscribing compressive-forces, so as to (iii) augment return-circulation of depleted-fluids from body tissues of the lower-leg extremities. Those compressive-focus are applied in placement-areas, described herein, of the lower-leg-extremities which function to augment return toward the heart.

Those circumscribing compressive-forces initiate and implement return-circulation of depleted interstitial fluids and wastes. Such return-circulation compressive-forces, are applied where most needed and most helpful: starting from the leading forward end of the foot-arch; and, directionally-continuing those circumscribing compressive-forces for properly directing return toward the heart of the user.

Initiating and sustaining such removal of depleted fluids, and wastes, from the lower-leg extremities also significantly implements a complimentary function. That is facilitating arterial return of replenishing oxygen and nutrients to body-tissues of such lower-leg-extremities. That arterial-return of oxygen and nutrients would otherwise, would be impeded as a result of described periods of immobility.

Combinations which implement the therapeutic action of the present invention, include: (i) initiating circumscribing compressive-forces at the forward-leading edge portion of the foot-arch, (ii) extending those forces directionally through the full length of the foot-arch; (iii) directionally-continuing such compressive-forces onwardly through an enlarged placement-area, surrounding the complex-configuration cross-section which includes the individual's heel; so as to: (iv) directionally-continue those compressive-forces through a placement-area contiguous to the individual's ankle area; while: (v) specifically, limiting application of such compressive-forces to no-more than approaching the lower-portion of such a user's calf-muscle, for each leg.

The therapeutic sock of the invention specifically avoids any compressive-forces on the calf muscle, thigh muscle, or any above-located leg muscle of a user. The invention is not directed to: (i) arch support or (ii) athletic-support endeavors of any kind. That is: the identified functional relief purposes differ entirely from support or athletic endeavors. The relief provided by the invention continues following a period of immobility, results from consecutive usage over a few days during periods of immobility eliminates those difficulties entirely.

The beneficial effects of usage of the special elasticized fabrics of the therapeutic-sock of the invention, are readily evident to an individual user following a period of immobility. Those beneficial effects are pronounced when such an individual does initiate walking-about following such a period of immobility. Previously-experienced difficulties, following such a period of immobility, are no longer experienced. That is, such a user does not “hobble” when walking-about following such immobility. As pointed-out continuing appropriate usage eliminates all discomfort in the lower-leg-extremities. Further, by making the arterial return of oxygen and nutrients augments the desired return-circulation toward the heart, for such an individual user.

Placement-areas, for work of the invention, on lower-leg locations are identified in the schematic presentation.

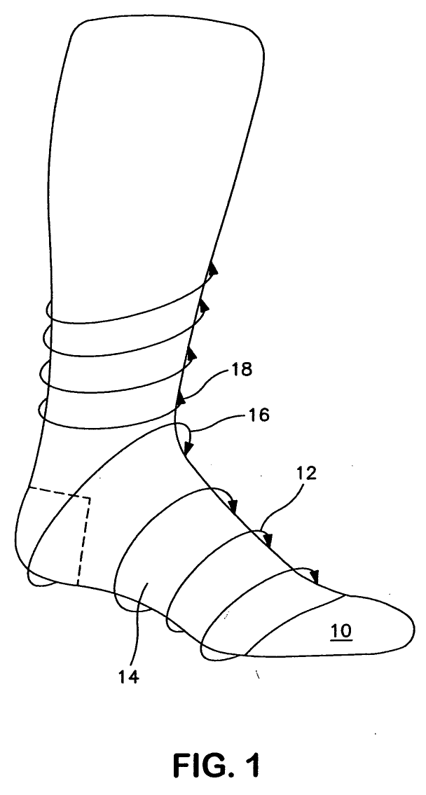

A relatively-thin “soft” toe area 10 for a sock-embodiment of the invention is provided in FIG. 1. That thin-fabric toe area does not circumscribe compressive-forces. FIG. 1, toe area 10 is fabricated to avoid bulk which could contribute an increased the cross-sectional size, at such wide part of the foot where the five toes join the metatarsals. The soft fabric of toe-area and its positioning, help to properly locate the foot in a therapeutic sock of the invention.

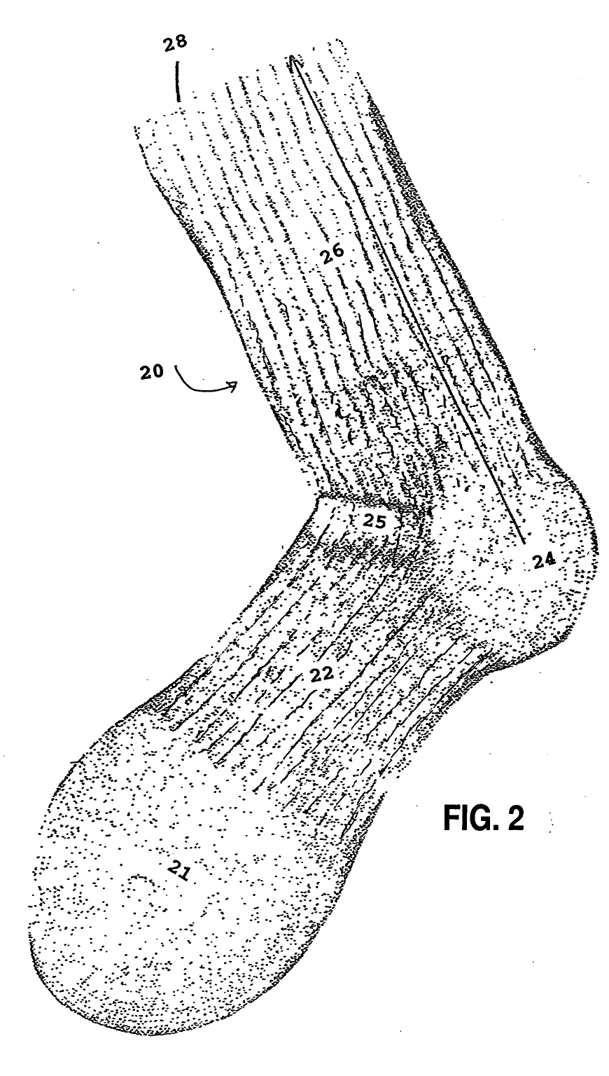

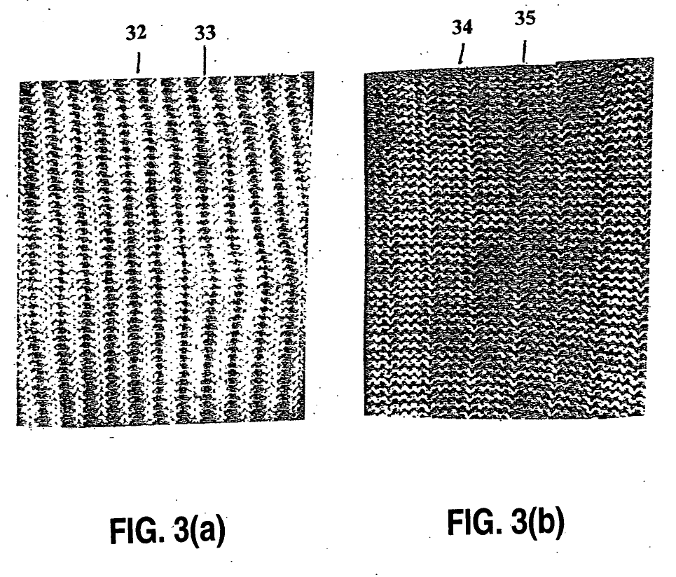

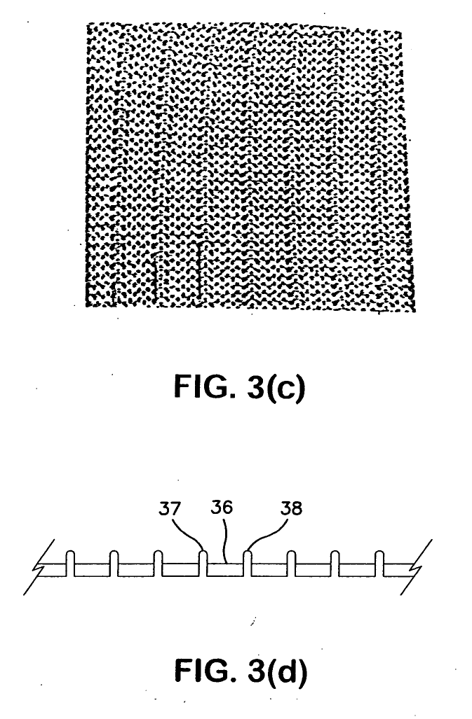

Circumscribing compressive-forces rely largely on a thick- elasticized-fabric of the invention, which predominates in FIG. 2; and, is described in more detail in relation to the enlarged views of FIGS. 3(a) through 3(d). Such thick-elasticized fabric generates its compression during expansion which occurs when invention is put on by a user. Then circumscribing compressive-forces; which are initiated at the forward portion entering the foot-arch. Those circumscribing forces are as indicated by circumscribing-ring 12 of FIG. 1, which is located centrally of the foot-arch. Those compressive-forces of the invention extend throughout the length of the foot-arch; which is designated generally by 14, in FIG. 1. The effects of such compressive-forces are enhanced throughout the lower-leg extremities by special measures; including maximizing use of such thick-elasticized fabric by directional-continuity on each sequential designated placement-area of a user's lower-leg extremities. That sequential directionally-continuing application of circumferential compressive-forces as described herein, provides intended return circulation of depleted-fluids from such extremities.

Special fabricating measures, including combining thick and thin elasticized fabrics, enables continuing such compressive-forces through a complex cross-sectional configuration enlarged surface placement-area. The location for that enlarged area is indicated generally by ring 16 of FIG. 1. That enlarged placement-area, which is described in greater detail later herein, includes the heel; and, the circumferential dimension at that location increases by about one-third, or more, in relation to the circumference at location 12 of foot-arch 14.

Special elasticized fabrics of the invention are combined for covering that enlarged placement-area, indicated by ring 16 of FIG. 1. Fabrics of differing configurations, and elastic strengths, are combined in that enlarged placement-area in the therapeutic sock of the invention. A thin acrylic-like elasticized fabric covers a placement-area for the heel, which is shown by interrupted lines. That thin fabric is combined with wide-rib thick-elasticized fabric of the invention; which is located forwardly of the heel. That combination of thin and thick elasticized fabrics contributes significantly to providing for directionally on-going compressive-forces in that enlarged area. The thin elasticized fabric covers the lower surface, the left and right side surfaces, and the back surface of the heel, while the thick wide-rib fabric covers the placement-area frontally of the heel.

A further contribution is that such thin elasticized fabric for the heel also functions to provide ease-of-usage and proper-placement in putting on a sock-embodiment of the invention; and, later, contributes to properly-locating the foot, within a shoe when such sock-embodiment of the invention is properly in place, within a shoe.

That concept of combining thick and thin fabrics, assist3s in directionally-continuing, and conveniently maximizing, compressive-forces throughout that enlarged placement-area, with the complex cross-section indicative by 16 in FIG. 1, which includes the heel. That accomplishment by a combination of fabrics, further contributes to directional-continuity into a placement-area which, as later described in more detail, includes the ankle of a user. That area is indicated designated 18 in FIG. 1. As taught herein, a limitation leg-height contiguous to such placement-area for the ankle, prohibits circumscribing compressive-forces from extending height-wise beyond approaching the start of a user's calf muscle.

In accordance with the invention, no circumscribing compressive-forces of the invention are applied to the calf muscle, thigh muscle, or, any such upper-leg muscle of the user. However, a “soft-entry” for the foot of a user can be provided at such calf muscle location. It is to be understood that any covering, extending height-wise above that lower approach to the calf muscle, must be free of the described circumscribing compressive-forces of the invention.

In FIG. 2, a view of sock 20 of the invention is presented; shown non-expanded; that is, when not in use; and sock 20 lies flat. The special elasticized fabrics of sock 20 are not expanded at any location. Non-expanded, side-by-side, linearly-extended, rib-like-sections of the specialized thick-elasticized fabric, start contiguous to soft-fabric toe portion 21 at FIG. 2; and, that large-rib the thick-elasticized fabric, extends from toe portion 21 throughout the full foot-arch placement-area, which is designated 22.

Part of the concepts for optimizing covering the sequentially-next, complex cross-section, enlarged-surface placement-area, which includes heel 24, comprises combining two elasticized fabrics of the invention. A relatively-thin elasticized Spandex or acrylic type fabric is used in placement-area 24; that is the bottom surface, left side surface, right side surface, and back surface of heel 24, are covered by such relatively-thin elasticized fabric.

That thin elasticized fabric facilitates; (i) optimal placement of the foot within a sock embodiment of the invention; (ii) helps to contribute to directional-continuity for circumscribing compressive-forces of the invention; and, further (iii) helps to facilitate putting on a shoe while maintaining the sock-embodiment properly in place for such directional-continuity.

Use of a thin elasticized fabric at placement-area 24, contributes to: (i) directional-continuity of circumscribing compressive-forces throughout, and following, such complex cross-sectional configuration enlarged-surface placement-area, where the foot joins the leg. That is, such concepts contribute to: (ii) enabling location of a major portion; for example about sixty (60)% of the thick-fabric large-ribs, in placement-area 25 of FIG. 2, frontally of heel placement-area 24. Numerically, about two thirds of the large-rib thick-fabric ribs, circumscribing foot-arch 22, extend frontally of such-heel placement-area 24.

Such frontally-located placement-area 25 is inter-connected around the frontal side-edge portions of the thinner Spandex-type fabric for the heel placement-area 24; while remaining edge portions of the side walls and back wall, of the heel, are directionally interconnected, in FIG. 2 to ankle surface-area 26. The latter continues a circumferentially-continuous large-rib thick-elasticized-fabric, such as that of the foot-arch (22), combined-fabric area.

That arrangement and the special combinations of elasticized-fabrics contribute to extending and maximizing ongoing circumscribing compressive-forces; and, contribute to more uniformly maximizing continuing return-circulation compressive-forces for depleted body-fluids throughout the placement-areas contacted by the therapeutic sock of the invention. Those proportional combination concepts help to provide described benefits of sock-embodiments of the invention to users who, individually, may have differing shoe, and resultant sock, sizes.

Circumscribing compressive-forces are achieved by such combination-concepts when sock 20 is expanded; that is: by being put into use. However, the circumscribing compressive-forces of the invention are diminished, and eliminated, in approaching the lower portion of a user's calf muscle. That is, such compressive-forces in placement-area 26 of FIG. 2, for the ankle, are purposefully limited to a contiguous height-wise area which does not cover any portion of a user's calf muscle. Compressive-forces in placement-area 26 of FIG. 2 can extend toward 28; that is, approaching the lower calf-muscle level for a user. The length of that height-wise-extension can be limited to a “low-cut” style embodiment so as to work satisfactorily, during relatively short time-periods for certain sports. In general however, height-wise extension is dependent on the person's height and sock size, and, provides circumscribing compressive-forces extending toward a lower calf muscle area, designated as 28 in FIG. 2.

Soft fabric, cotton or cotton combined with nylon, can be located at the lower calf-muscle area for a user; so as to define a soft-entrance opening for the foot at such level. That is, the described circumscribing compressive-forces of the special wide-rib thick elasticized-fabric are terminated in approaching level 28 of FIG. 2; at which, a “soft-entry” area for a user's foot can be provided.

Useful leg-height dimensions for a sock-embodiment, measured from the center of the heel placement-area 24 of FIG. 2, can start at about five inches, depending on the height of a female or male user. However, present teachings purposefully eliminate circumscribing compressive-forces, as described herein, or the calf muscle or any leg-muscle that approach to the lower portion of the calf muscle of the user. As taught herein, extending such compressive-forces above such limits would tend to diminish the useful return-circulation, which has been enhanced by the actions of the invention on placement-areas of the lower-leg extremities.

In the non-expanded flattened top-surface view of the sock embodiment of FIG. 2, the dimensions for such top surface area, as shown, are essentially the same, as the dimensions of the bottom surface-areas in the therapeutic sock of the invention.

However, an enlarged external view of thick-fabric ribs shown in FIG. 3(a), in a non-expanded minor-surface-area; from for example, placement area 26 of FIG. 2. That non-expanded minor-surface-area presents twelve elongated-ribs of the selected thick elasticized-fabric of the invention. The widthwise dimension of ribs for such thick fabric can be selected; and, thick-fabric ribs are utilized throughout foot-arch 22 of FIG. 2 and, as described, extend frontally of the thin-elasticized fabric covering heel placement area 24. Such thick-fabric ribs in placement-area 25, frontally of placement-area 24, extend into the uniformly thick-fabric ribs of placement-area 26 of FIG. 2. The latter includes the ankle of such user; and, thick elasticized fabric can extend onwardly toward the lower-level of the user's calf-muscle.

Those thick elasticized-fabric ribs are continuous, around the full-circumference of, and along the length of, the placement-area for the arch, designated as 22 in FIG. 2. Such thick-elasticized fabric extends around the full circumference of placement-area 26 for the ankle, as shown in FIG. 2; with the thick-fabric frontally of the heel, at placement-area 25, contributing to quantitatively optimizing on-going circumscribing compressive-forces through that enlarged placement-area.

The enlarged detailed view of FIG. 3(a) shows non-expanded ribs in a minor-area portion of such thick elasticized-fabric taken, for example, from placement-area 26. Non-expanded ribs 32 and 33 of FIG. 3(a) are separated by two intermediate non-expanded ribs. That minor portion presentation of thick fabric occurs before the sock is expanded by being put into use. That circumferential external-surface-portion, of a minor selected portion of the thick fabric, as shown enlarged in FIG. 3(a), presents twelve side-by-side, linearly-extended, non-expanded ribs. Rib-size throughout the thick-fabric areas of the invention, can be selected for larger sock-size embodiments so as to provide substantially thick-fabric rib-size throughout such larger size embodiment.

FIG. 3(b) shows an elevational view of such exterior-surface minor-portion of FIG. 3(a) which results from putting the sock into use. That expands the elongated ribs of thick-elasticized fabric widthwise. The non-expanded minor-surface portion of FIG. 3(a), presents twelve ribs. Note, however, that when such minor-portion is expanded, during usage, that as shown in FIG. 3(b) such minor-portion now presents seven contiguous side-by-side expanded ribs. That relationship, which can be selected, helps to quantify a ratio for the circumferential compression-force. In place of that twelve non-expanded ribs, which had occupied such fixed minor-surface portion, is now occupied by seven expanded ribs, as the result of putting the sock embodiment into use.

The selected ratio, such as twelve to seven (12/7), provides a quantified indication of the compressive-force exerted circumferentially by that selected rib-size for the thick-elasticized-fabric of the invention. The compressive-force is also, in-part dependent on the cross-sectional dimension of a designated placement-area; that is, the ratio of widthwise expansion, during usage, is based in-part on the cross-sectional size of the lower-leg-extremity placement-area where such fabric is being used. Such compressive-force could increase, for example, in that dimensionally-enlarged placement-area for the lower-leg extremity which includes the heel; such an increase would, however, be at least partially offset by thin fabric covering the heel 24 so as to provide a near uniform circumferential compressive-force in that area.

A representative circumference around the foot-arch can average around ten inches. However, that increase in circumference for the enlarged placement-area which includes the heel is dependent on the user; and, the circumscribing compressive-force in that area is tempered by the thin-fabric of the heel, as referred to above.

The circumference for placement-area 26 of FIG. 2, contiguous to a user's ankle, can average only slightly above ten inches, dependent on the user. However, as taught herein, any such circumscribing compressive-forces are eliminated in approaching the lower portion of a user's calf muscle. That is achieved by selections and limitations on the proper leg-height; as described herein as part of the invention. While softer fabrics can be introduced as a desired height is approached, with the objective to eliminate any such circumscribing compressive force on the individual's calf-muscle.

In accordance with the invention, the level of elasticity is coordinated for placement-areas, as described; however such sizes could be changed to take into account the differing sizes for accommodating women and men. Also, selected types of elastic yarn, for example, those using polymeric fibers can be selected to have the elasticity changed by treatment, such as, by annealing the fabric.

Stretchable fabric constituents, for example, can include polyurethane-cross-linked Spandex and Lycra thin fabrics which are used, in particular, for the thin sides, bottom, and back surface areas defining the heel. Other polymers produced with desired elastic characteristics, such as polyester, can be used in the thick-elasticized-fabric portions; thus enabling fabrication of sock-embodiments adaptable to the particular age, or need, of the user. Various combinations of cotton and nylons can be used for “soft” toe and entry portions. Also, various combinations of newly-developing polymers, treated and untreated, by following the present teachings can effectively eliminate the difficulties or discomfort associated with the lower-leg extremities, which, prior to the present invention, had resulted from defined relevant time periods of immobility.

FIGS. 3(a) and 3(b) depict external-surface views of a minor portion of the thick-elasticized fabric. However, FIG. 3(c) shows an expanded interior-surface view in elevation, of such minor-portion; which is expanded so as to depict open-faced widened-channels which augment return-circulation of depleted body-fluids from the lower-leg extremities.

And, in the cross-sectional view of FIG. 3(d), such interior open-faced widened-channels are shown as supported by channel-support portions, located to provide support by surface-contact, of those elongated channel-support portions with the user. The combined area of a pair of those surface contacts with the user, is significantly less than the width-dimension across a single open-faced widened-channel. Such relationship, of minimized surface-contact support width to open-faced widened-channel width continues throughout the interior contact of the placement-area; such as foot-arch, or the ankle placement-area; with both utilizing the described thick elasticized fabric of the invention. And, as described above, also continues through the thick-fabric major-portion, frontally of heel 24 in that complex cross-section enlarged placement-area of FIG. 2, which uses the thin elastic fabric at heel 24.

FIG. 3(d) is a cross sectional view, such thick-elasticized fabric, in a plane which intersects, substantially-perpendicularly, with the central-axis of the placement-area. An expanded-usage portion is shown, in which stretched elasticized thread 36 extends circumferentially of, for example, the ankle placement-area. Selected thread 36 as stretched, extends between surface-contact supports, such as 37, 38, which contact the surface at the respective placement-area of the user. The elasticized-threads are continuous around the full circumference of the placement area; but, they stretch across and, are spaced from, each open-faced widened-channel, as shown in FIG. 3(d).

The longitudinal spacing of those stretched elasticized-threads, such as 36, be determined by selection of the thick fabric to be used; and, remains about the same in placement-areas for a selected thick-elasticized fabric, such as described earlier. For example: about twenty-four threads, such as 36, exist per inch of longitudinally-extending dimension; which amount to about nine point four such threads per centimeter, in the height-wise return-direction in the ankle-placement-area.

Those elasticized-threads extend between surface-contact supports such as 37, 38 of FIG. 3(d). Those surface-contacts longitudinally-extending also act as guides for depleted-fluids being retrieved along intermediately-located width of the widened channels; at which no surface-contact with the user is made ordinarily during such return function.

Another advantage such circumferentially continuous of such elasticized-threads of the thick fabric, as taught herein come into play should a user purchase a pair of socks of the invention, which are found to extend the intended compressive-force height-wise leg-length extends into the calf muscle. In that situation, the leg-length can be shortened to a desired height by horizontally cutting, with standard scissors, to a desired leg-length height for the sock. That is: the circumferential-continuity of such elasticized threads (such as 36, FIG. 3(d)) prevents unraveling of the fabric at the cut height.

Surface-contact, edge-supported, spaces confronting a placement-area, provide open-faced widened-channels for augmenting return-circulation of depleted fluids from body-tissues of the lower-leg-extremities, for return circulation toward the heart. Such open-faced widened-channels, are the result of stretching the thick elasticized-fabric ribs at the described placement-area; including, that thick-fabric placement-area frontally of the heel, in circumscribing that complex cross-section enlarged placement-area. That is, by utilizing the described combination of such thick-fabric large-ribs with the smaller-rib thin-fabric smaller-rib for the placement area of the heel, helps to maintain desired circumscribing compressive-forces through such enlarged surface area.

The elongated surface-contacts on each side of an open-faced widened-channel help to provide return directionally. And, that directional-continuity is also implemented by the thin elasticized-fabric, which provides smaller channels at the sides, bottom and back surfaces of the heel, as shown at 24 in FIG. 2.

The circumferential distance across a thick-fabric open-faced widened-channel can be selected in a range of about two times, to about three times larger, than the combined area of support-surface contacts, on opposite sides of a widened-channel; and can, preferably, be achieved by dimensional selection of the rib-size for the thick-fabric.

Locating such surface-contact on each side of a widened-channel, as shown in FIG. 3(d), contributes to the desired return directional-continuity effect of the compressive-forces. Further, such surface-supports, for such widened channels, are adaptable to the particular-contour of the placement-area on the user's lower leg extremities, where such support is provided. For example, contiguous to the ankle of a user, each open-faced widened return-circulation channel can have a cross-width dimension of above one-fourth inch, measured in the plane of the circumscribed surface-area; while, surface-support contact on the flattened surface each side of such ankle, can be less than that amount, as measured in a surrounding substantially-flat plane. That ratio of widened-channel dimension to support-surface contact dimensions, can be selected in a range, from about two-to-one extending to about four-to-one, dependent on selections of rib-size for the thick-fabric.

In addition to directional-continuity, reasonably uniform return-capabilities in contiguous placement-areas is important to continuity. Also, decreasing the surface-area for support contacts can augment the directional return by the thick-elasticized fabric large-rib open-faced widened-channels of depleted body-fluids from the lower-leg extremities; while taking into account that guiding of such return is augmented by such surface-contacts.

Referring to FIG. 2, an average dimensional height-wise range, for most users, from the center of heel 24, to a selected leg-height-dimension, which approaches the lower-end of the calf muscle, could be in a range of about five inches to approaching eight inches; with that dimension being largely dependent on the height of the user. That length can be selected prior to use; or, can be cut to desired length because of the circumferentially-continuous elasticized-thread, as described, which will prevent unraveling when cut to a desired height. The height of entrance-opening for the sock is preferably selected to be slightly less than a dimensional height contacting the user's calf-muscle, when the sock embodiment is in use.

Present teachings recognize that the capillaries carry-out functional purposes of the blood-circulation system. In proper operation of that system, the volumetric capacity of capillaries, for such body-tissues, should remain about equal to the capacity of the supplying arteries and the return veins. The main supply arteries branch repeatedly to form arterioles which feed capillaries supplying body-tissues of the lower-leg extremities. That supply has an arterial-pressure capable of readily entering capillaries of the lower-leg extremities, for supplying oxygen and nutrients to cells of those body-tissues. However, resultant depleted-fluids, with absorbed carbon dioxide and other wastes, can present return difficulties for the above-described individuals. Other capillaries of the lower leg extremities should return depleted fluids. That is, carbon-dioxide and other wastes of those fluids, should normally be returned by those capillaries and directed for return-circulation.

In practice however, there can be a tendency for lower-leg extremities, of described potential-users, to swell. For example, during seated-immobility of an extended air flight. As a result, functioning of those return capillaries can be impaired, such that depleted interstitial-fluids tend to dwell in spaces around such body-tissues.

However, circumscribing compressive-forces, as taught herein, positioned on the foot-arch, and directionally-sequential placement-areas of the lower leg extremities, as shown and described, help to overcome that “dwelling” problem by augmenting proper return circulation of such depleted fluids.

The therapeutic sock embodiments of the invention, being described are not for foot-arch support; nor for any athletic support of the legs. And, none apply compressive forces to the calf muscle, the thigh muscle or, any such upper leg-muscle of the user.

The circumscribing-compressive-forces of the invention help to maintain appropriate return-circulation of depleted body-fluids, from the lower-leg extremities, for proper return, especially during periods of substantial-immobility and following such periods of immobility. The desired return-circulation is initiated by starting circumscribing compressive forces at the forward leading edge portion of the foot-arch; and, extending those circumscribing compressive-forces directionally-onwardly, and sequentially, through the lower-leg extremities, as described.

Selective materials for fabrics of the therapeutic sock of the invention can utilize cotton and/or nylon in the soft toe and for soft foot-entry purposes. And, utilizes thin elastic polyurethane Spandex or Acrylic fabrics, at locations for the thinner “bottom, sides, and back” of heel areas, as described herein. Synthetic fibers which provide desired elasticity; are available from manufacturing companies, such as: DuPont Co., Wilmington, Del., and Eastman Chemical Company of Kingsport, Tenn.

Selective individual polymeric synthetic fibers can include:

polyurethane derived Spandex and

acrylics, sold commercially as “Acryla”, and

elasticized polyesters.

Further, synthetic-polymer fabrics can have inherent properties influenced by the spinning; and, also, by post-spinning treatments of the polymers, including annealing or other finishing, so as to increase strength, stiffness, or elasticity. Examples are set forth in the accompanying tabulation; and, the following written description.

| Elasticity Rating | Name of Fabric | Preference |
|---|---|---|
| High | Acrylic/polyester | Arch, Heel, and lower-leg |
| Medium | Polyester | Approaching calf-muscle |
| “Soft” Low | Cotton or nylon, | For toe area and foot- |
| | or combinations | entry portion |
| | thereof. | |

A thick-fabric of elasticized polyester is used in the foot-arch, in the frontal-passage of the heel area, and contiguous to the ankle area of the lower-leg extremities; free of contact with the user's calf-muscle. Other polymeric elasticized materials for fabrics; and, additive treatment of threads and fabrics for improvement in performing specific functions, help to develop suitable characteristics, as described above, can be selected for proper functioning.

A written description of the present invention, and of the manner and process of making and using it, with specific details as to fabric materials, methods of sock fabrication, and arrangements controlling functioning, have been set-forth so as to enable those, who become skilled in the technology newly-disclosed herein, to make and use the present invention. The patentable scope of such invention is set forth in the claims and, the language of those claims is to be interpreted in the light of the above disclosure.

What is claimed is:

1. Therapeutic-sock fabricating method for increasing supply of oxygen and nutrients to lower-leg-extremities of an individual so as to avoid difficulties associated with confinement to substantial immobility, for relevant time-periods, comprising
A) fabricating elasticized fabric for applying circumscribing compressive-forces on designated sequentially-located placement-areas of the lower-leg extremities of an individual user, by
(i) initiating such compressive-forces at the forward leading-edge of the foot-arch of such individual user, and
(ii) continuing such compressive-forces to extend directionally throughout such foot-arch area, while
B) continuing applying such compressive-forces on directionally-sequential placement-areas of the lower-leg extremities of such individual, as follows:
(i) an enlarged placement-area, which includes such individual's heel, then
(ii) onto a placement-area contiguous to such individual's ankle, while
(iii) avoiding extending such compressive-forces, on the user's leg, beyond merely approaching a lower-portion of such individual's calf-muscle.

2. The Invention of claim 1, in which
C) continuing such circumscribing compressive-forces directionally in such enlarged area, which includes the individual's heel, by selectively combining elasticized-fabrics for such enlarged placement-area, so as to enable
D) extending beneficial effects of selected elasticized-fabrics, as directed onward toward veins in each leg of the individual user, which provide for one-way return toward the heart of such user.

3. The Invention of claim 2, including
E) fabricating a thick elasticized-fabric, for
(i) surface-contact with designated placement-areas of the lower-leg extremities of the user, for
(ii) establishing open-faced widened-channels, by such surface-contact, which augment return of depleted body-tissue fluids from such lower-leg extremities; by
F) supporting such open-faced widened-channels confronting such placement-areas, by
(i) providing-supporting side-wall surface-contacts for such widened-channels, disposed along each elongated side of such a channel, with
(ii) such sidewall-contacts as oppositely-disposed along elongated sides of sub widened channels, augmenting provision of:
(iii) widened-channels spaced by such surface-contacts with such user by such elongated side-wall supports.

4. The Invention of claim 3, in which
G) such an elongated pair of side-wall supports occupy a lesser combined-area than that of such an intermediately-disposed open-faced widened-channel, which enables return-circulation space in the range of about two to about four times greater, than that of the combined surface-area contact of such respective oppositely-disposed side-wall support contacts.

5. The invention of claim 3, in which
I) supplying blood by arteries of such user is directed for
(i) replenishing oxygen and nutrients, by
(ii) entering capillaries in body-tissues of the lower-leg extremities, and, in which
J) removing of depleted interstitial fluids from body tissues of the lower-leg extremities of such user, is achieved by
(i) augmenting recirculation of such fluids, by
(ii) responding to such circumscribing compressive-forces on lower-leg extremities, establishing such open-faced widened-channels, for
(iii) facilitating removal of depleted interstitial-fluids and wastes, with directional guidance, augmented by
(iv) providing such elongated support-surface-contacts for such depleted-fluids toward vein locations of such user providing for movement toward the heart.

6. The invention of claim 5, further including
K) implementing such removal of depleted interstitial-fluids from body-tissues of the lower-leg extremities of such user; by
(i) replenishing supplies of oxygen and nutrients through arteries, of such user, with sequential
(ii) branching into arterioles, and, sequentially into
(iii) numerous incoming thin-walled capillaries, which
(iv) supply oxygen and nutrients to cells of body-tissues of such user's lower-leg-extremities.

7. Therapeutic-sock, for use by men or women, fabricated to minimize lower-leg extremity difficulties resulting from substantial-immobility without opportunity to walk-freely about for a time-period, the duration of which can be dependent, at least in-part, on approaching or reaching seniority for such an individual user, comprising
A) a soft-fabric toe-cover, then providing
B) a thick elasticized fabric,
(i) capable of generating compressive-forces, due to circumscribing expansion when such sock is put into use, starting:
(ii) at the forward leading edge of the foot-arch of such individual user; and, continuing-directionally
(iii) through the full-length of the foot-arch; then, onto
C) a complex cross-sectional configuration defining an enlarged placement-area, which includes a placement-area for a user's heel; then, onto
D) a placement-area for the ankle, and, contiguous thereto of such user, while
E) limiting leg-wise height to terminating such compressive-forces in approaching a lower portion of such user's calf-muscle.

8. The invention of claim 7, in which
F) such heel placement-area utilizes a thin-spandex-type of elastic fabric, while extending such
(i) thick-elasticized-fabric, with lengthwise extending large-ribs, which,
(ii) expand circumferentially during-usage on a placement-area of such user,
(iii) creating compressive-forces which augment return of depleted body-fluids from the lower-leg extremities,
(iv) starting at:
(a) such leading-edge of the foot-arch, and
(b) continuing-directionally throughout the length of such foot-arch; then, continuing by utilizing
(c) a combination of thick and thin elasticized fabrics for such enlarged placement-area which includes the heel; with
G) combined thick and thin fabric compressive-forces acting on such enlarged placement-area to directionally-continue such compressive-force movement of depleted body-fluids, onto
(i) a placement-area surrounding and contiguous to the ankle, while
(ii) limiting heightwise leg-surface contact of compressive-forces to merely approaching the lower-edge of the calf-muscle of a leg of such user.

9. The Invention of claim 8, in which
H) such removal of depleted body-fluids from the lower-leg extremities of such user facilitate arterial return of oxygen and nutrients; to
(i) a plurality of thin-walled capillaries for replenishing oxygen and nutrients in body-tissues of lower-leg extremities of such user.

10. The Invention of claim 9, in which
I) such combination of fabrics for such enlarged placement-area, which includes the user's heel,
(i) position thick-elasticized-fabric frontally of such user's heel, and, in which
(ii) such thin spandex-type of elastic material, covers
(a) the bottom, two sides, and back surfaces of such heel, so as to combine with
(b) such frontally-positioned thick-elasticized-fabric, for
(c) extending such circumscribing compressive-forces through such enlarged placement-area, toward such lower-portion of the user's calf muscle.

11. The Invention of claim 10, in which
I) such thin-fabric heel placement-area, as used in combination with such frontally-positioned thick-fabric;
J) facilitates:
(i) properly locating a user's heel in such thin-fabric heel placement-area for such sock, plus
(ii) eases establishing proper orientation for the heel of such user's foot when putting on a shoe.

12. The Invention of claim 11, in which
K) such thin-fabric heel placement area also facilitates properly-locating compressive-forces, as supplied sequentially on placement-areas of such user's lower-leg extremities, for directing depleted-fluid for return through leg-veins toward the heart of such user.

13. The Invention of claim 12, in which
L) such circumscribing compressive-forces, starting with the foot-arch,
(i) impel return of depleted interstitial fluids from the body-tissues of lower-leg extremities of the user, so as to enable
(ii) arterial-supplied fluids to replenish oxygen and nutrients within such body-tissues of such user.

14. The Invention of claim 13, for use by
M) an individual when confined to substantial immobility for an extended time interval, such as: by
(i) an extended air flight, without
(ii) opportunity for walking-freely about.
